(19) United States
(12) Patent Application Publication
(10) Pub. No.: US 2024/0319566 A1
(43) Pub. Date: Sep. 26, 2024

(54) AUXILIARY STARING AND IMAGING FOCUSING DEVICE

(71) Applicant: Crystalvue Medical Corporation, Taoyuan City 330 (TW)

(72) Inventors: Yen-Jen CHANG, Taoyuan City (TW); Chung-Ping CHUANG, Taoyuan City (TW); Chun-Nan LIN, Taoyuan City (TW)

(21) Appl. No.: 18/606,306

(22) Filed: Mar. 15, 2024

(30) Foreign Application Priority Data
Mar. 23, 2023 (TW) 112110981

(51) Int. Cl. G03B 15/06; G03B 3/04; G03B 11/04

(52) U.S. Cl. CPC G03B 15/06; G03B 3/04; G03B 11/045; A61B 3/0008

(57) ABSTRACT

An auxiliary staring and imaging focusing device includes an illumination system, an imaging system, a staring device and a focusing device. The illumination system has an illumination optical-path to project a detection light to illuminate a fundus of a subject's eye. The imaging system has an imaging optical-path for receiving a reflected light and a fundus image of subject and imaging the reflected light and fundus image on an image display. The staring device is located in the illumination optical-path and forms a staring surface provided with staring points contrast with a detection light for the subject to watch. The focusing device is provided with a split-image screen in the illumination optical-path. The split-image screen has a shutter with a default size, and two facing prisms are disposed on the shutter and a light-transmitting space is formed between two prisms for a center staring point to pass through.

### BACKGROUND OF THE INVENTION

### 1. Field of the Invention

The invention relates to an imaging device; in particular, to an auxiliary staring and imaging focusing device.

### 2. Description of the Prior Art

Referring to FIG. 1, in the conventional auxiliary staring and imaging focusing device 1, the split-image screen 12 has a shutter 120 with a default size, and the shutter 120 is provided with a light-transmitting slit 123, and the front row of the light-transmitting slit 123 is provided with two opposite prisms 121˜122. The staring surface 14 is provided with a plurality of staring points 10 that contrast with the detection light. The split-image screen 12 and the staring point 10 form an opaque area blocking the illumination light path, and a light-transmitting area is formed by the blocks outside the opaque area.

However, as shown in FIG. 2 and FIG. 3A to FIG. 3C, it can be known that no matter which relative position of the two prisms 121˜122 is in FIG. 3A to FIG. 3C, if the light-transmitting slit 123 is too narrow, there will be a problem that the central staring point cannot pass through the light-transmitting slit 123 between the two prisms 121˜122, which needs to be further solved.

### SUMMARY OF THE INVENTION

Therefore, the invention provides an auxiliary staring and imaging focusing device to solve the above-mentioned problems of the prior arts.

A preferred embodiment of the invention is an auxiliary staring and imaging focusing device. In this embodiment, the auxiliary staring and imaging focusing device includes an illumination system, an imaging system, a staring device and a focusing device. The illumination system has an illumination optical-path to project a detection light to illuminate a fundus of a subject's eye. The imaging system has an imaging optical-path for receiving a reflected light and a fundus image of the subject and imaging the reflected light and the fundus image on an image display. The staring device is located in the illumination optical-path and forms a staring surface provided with staring points contrast with a detection light for the subject to watch. The focusing device is provided with a split-image screen in the illumination optical-path. The split-image screen has a shutter with a default size, and two facing prisms are disposed on the shutter and a light-transmitting space is formed between the two prisms for a center staring point to pass through.

In an embodiment, the light-transmitting space is formed by separating the two prisms from the middle of the two prisms.

In an embodiment, the light-transmitting space is formed by replacing the middle of the two prisms with a plane.

In an embodiment, the two prisms are made of plastic material, and the light-transmitting space is formed by digging a hole in the middle of the two prisms.

In an embodiment, the illumination system is provided with an additional light-emitting unit set as a light-emitting diode, a lamp, a light bulb or an optical fiber.

In an embodiment, the staring point and the split-image screen are located on the same staring surface, and the staring surface is set as a vertical plane, and the staring point is located at set positions in four directions of up, down, left and right outside the split-image screen.

In an embodiment, the staring point and the split-image screen are located on the same staring surface, and the staring surface is set as a concave surface corresponding to the curvature of the human eye, and the staring point is located on the concave surface in four directions of up, down, left and right in front of the split-image screen.

In an embodiment, the detection light is a visible light, and the staring point is provided with at least one opaque-type light-shielding unit.

In an embodiment, the split-image screen and the staring point jointly form an opaque area blocking the illumination optical-path, and a light-transmitting area is formed by blocks outside the opaque area.

In an embodiment, the light-transmitting area is provided with a transparent substrate, and the split-image screen is disposed in the center of the transparent substrate, and the light-shielding unit is disposed at a set position on the transparent substrate outside the split-image screen.

In an embodiment, the detection light is an invisible light, and the staring point is provided with at least one light-emitting unit that can selectively control lighting or dimming, the light-emitting unit is set as a light-emitting diode, a lamp, a light bulb or an optical fiber.

Compared to the prior art, the auxiliary staring and imaging focusing device proposed by the invention has the following advantages and effects:
(1) directly integrating the staring and focusing device into the lighting and imaging system without the need to extend other optical-path systems, so the optical-path structure design of the fundus camera can be greatly simplified;
(2) designing an independently controllable staring point as a staring target to allow the subject to focus more on staring in a specific direction; and
(3) the shutter of the split-image screen is provided with two prisms facing each other and a light-transmitting space is formed between the two prisms for a center staring point to pass through. Therefore, the shortcoming in the prior art that the central staring point cannot pass through the slit on the shutter of the split-image screen can be effectively improved.

The advantage and spirit of the invention may be understood by the following detailed descriptions together with the appended drawings.

### BRIEF DESCRIPTION OF THE APPENDED DRAWINGS

FIG. 1 illustrates a schematic diagram of an auxiliary unit used in an auxiliary staring and imaging focusing device in the prior art.

FIG. 2 illustrates a schematic diagram of two prisms and slits disposed on the shutter of the split-image screen in FIG. 1.

FIG. 3A to FIG. 3C illustrate schematic diagrams of two prisms with different relative positions in the prior art respectively.

FIG. 4 illustrates a schematic diagram of an auxiliary staring and imaging focusing device used in a fundus camera in the invention.

FIG. 5 illustrates a schematic diagram of a first preferred embodiment of the auxiliary unit used in the auxiliary staring and imaging focusing device of the invention.

FIG. 6 illustrates a schematic diagram showing a light-transmitting space for the staring point to pass through formed between the two prisms disposed on the shutter of the split-image screen in FIG. 5.

FIG. 7A to FIG. 7C illustrate schematic diagrams of two prisms with different relative positions in the invention respectively.

FIG. 8 illustrates a schematic diagram of a second preferred embodiment of the auxiliary unit used in the auxiliary staring and imaging focusing device of the invention.

FIG. 9 illustrates a schematic diagram showing a light-transmitting space for the staring point to pass through formed between the two prisms disposed on the shutter of the split-image screen in FIG. 8.

FIG. 10A to FIG. 10C illustrate schematic diagrams of two prisms with different relative positions in the invention respectively.

FIG. 11 illustrates a schematic diagram of a third preferred embodiment of the auxiliary unit used in the auxiliary staring and imaging focusing device of the invention.

FIG. 12 illustrates a schematic diagram of a light-transmitting space for the staring point to pass through formed between the two prisms disposed on the shutter of the split-image screen in FIG. 11.

FIG. 13A to FIG. 13C illustrate schematic diagrams of two prisms with different relative positions in the invention respectively.

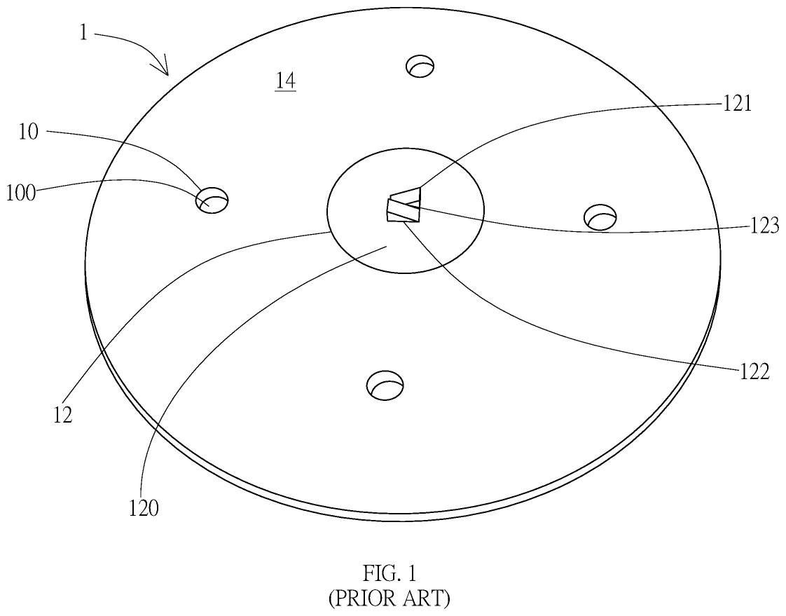

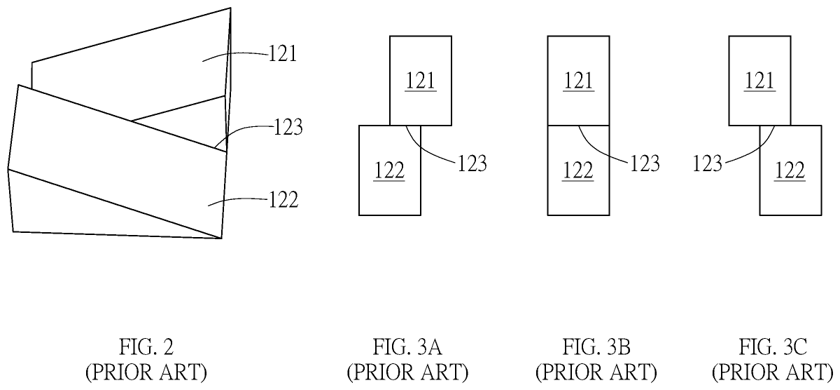

### DETAILED DESCRIPTION OF THE INVENTION

Reference will now be made in detail to the exemplary embodiments of the invention, examples of which are illustrated in the accompanying drawings. Elements/components with the same or similar numbers used in the drawings and embodiments are intended to represent the same or similar parts.

In order to facilitate the explanation of the technical features of the invention, the terms appearing in the following description are first defined. Unless otherwise specified, “system” mentioned in the following description refers to the lenses, lenses, reflectors, shields and assemblies of various other units; “optical-path” mentioned in the following description refers to the transmission path of light or imaging from the projection starting point (e.g., light emitter, eyeball) to the projection end point (e.g., eyeball, image display) in any system.

Since the auxiliary staring and imaging focusing device of the invention is directly integrated into a lighting and imaging system, there is no need to extend other optical-path systems to simplify the optical-path structure design of the fundus camera and a light-transmitting space can be formed between two prisms facing each other for the central staring point to pass through. In addition, an independently controllable staring point is designed as a staring target to allows the subject to focus more on a specific direction, thus effectively improving the deficiencies of the prior arts.

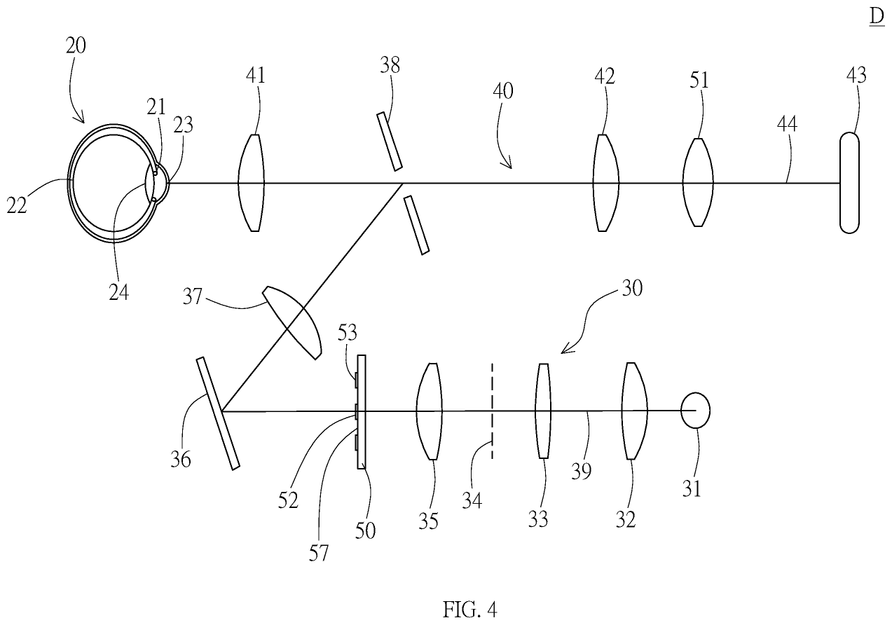

Please refer to FIG. 4. FIG. 4 illustrates a schematic diagram of the auxiliary staring and imaging focusing device used in the fundus camera in the invention. As shown in FIG. 4, the auxiliary staring and imaging focusing device D mainly performs the fundus 22 photography on the pupil 21 of the eye 20, and the auxiliary staring and imaging focusing device D includes a light emitter 31, a condenser lens 32, a first lens 33, a ring-shaped slit plate 34, a second lens 35, an auxiliary element 50, a mirror 36, a third lens 37, a perforate mirror 38, an ocular lens 41, a relay lens 42, a focal lens 51 and an image display (CMOS) 43, but not limited to this.

In the invention, the illumination system 30 is formed by the light emitter 31, the condenser lens 32, the first lens 33, the ring-shaped slit plate 34, the second lens 35, the mirror 36, the third lens 37, the perforate mirror 38; In addition, the imaging system 40 is formed by the ocular Lens 41, the relay lens 42 and the image display 43, but not limited to this.

In the illumination system 30, the light emitter 31 projects a detection light to the condenser lens 32 and the first lens 33, and an image is formed on the ring-shaped slit plate 34 and then projected to the mirror 36 through the second lens 35, and the direction of the light source is changed through the reflector 36, and the third lens 37 is used to drive the detection light onto the perforate mirror 38. Finally, the perforate mirror 38 reflects the detection light to illuminate the fundus 22 of the subject's eye 20 to form an illumination light path 39. By using a design of conjugate surface of the light emitter 31, the ring-shaped slit plate 34 and the pupil 21 of the eye 20, the aperture size of the image to the pupil 21 can be accurately controlled, thereby avoiding the effects of size difference or positional deviation of different light emitters.

In practical applications, the lighting system 30 can also be provided with an additional light-emitting unit, and the light-emitting unit can be set as a light-emitting diode, a lamp, a light bulb or an optical fiber, but not limited to this.

The imaging system 40 receives the subject's fundus image and the reflected light from the cornea 23 and crystalline lens 24 through the ocular lens 41, and under the action of the hole of the perforate mirror 38 and the relay lens 42, the fundus image and reflected light are displayed on the display 43 to form an imaging light path 44.

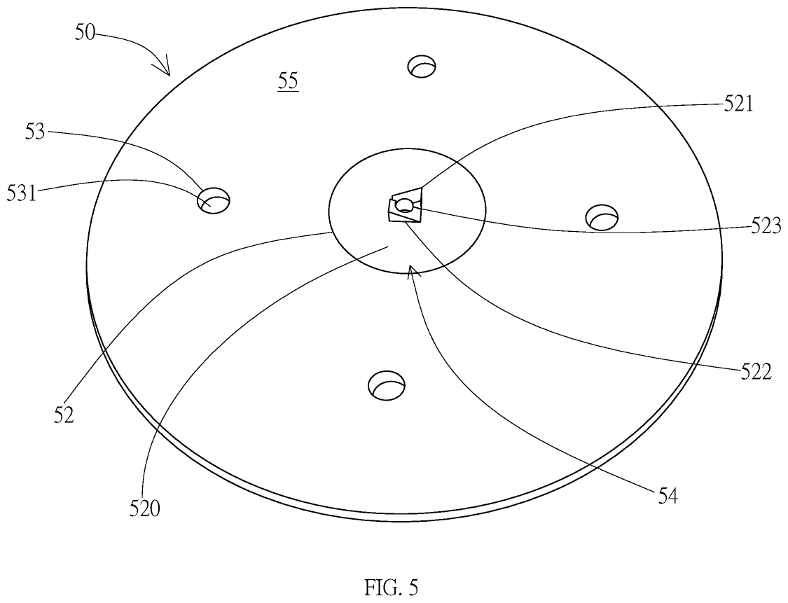

In addition, the auxiliary element 50 of the invention is disposed between the mirror 36 and the second lens 35 of the illumination light path 39. In the first preferred embodiment, as shown in FIG. 5, the auxiliary element 50 is provided with a split-image screen 52 and a plurality of staring points 53. The split-image screen 52 cooperates with the focusing lens 51 to form a focusing device generating split-image focusing, and the staring point 53 cooperates with the contrast of the detection light to form a staring device for the subject to rotate his/her eyeball to a specific required angle to respond the displayed staring target.

The split-image screen 52 has a shutter 520 with a default size. Two prisms 521 and 522 opposite to each other are disposed on the shutter 520, and a light-transmitting space 523 for the central staring point to pass through is formed between the two prisms 521 and 522; and the focusing lens 51 is located between the relay lens 42 and the image display 43 to form an adjustment means that can change the focus position. Thus, the detection light can pass through the light-transmitting space 523, and then change the direction of the detection light through the two prisms 521 and 522, and generate two rectangular images on the image display 43 to indicate whether the focus is completed, but not limited to this.

Furthermore, the split-image screen 52 and the staring point 53 are located on the same staring surface to form an opaque area 54 shielding the lighting system 30, and a light-transmitting area 55 is formed by the blocks outside the opaque area 54. The opaque area 54 is made of opaque material to avoid being penetrated by the detection light, while the light-transmitting area 55 is made of transparent material, so that the detection light can be projected into the subject's eyes. In fact, the staring surface can be set as a vertical plane or a concave surface corresponding to the curvature of the human eye, but not limited to this.

In this embodiment, the light emitter 31 includes a visible light source, the detection light adopts visible light, and the light-transmitting area 55 is set as a transparent substrate, and an opaque split-image screen 52 is disposed in the center of the transparent substrate. Four light-shielding units 531 are disposed at default distances outside the split-image screen 52 to form opaque staring points 53, wherein the staring points 53 are located at the upper, lower, left, and right directions outside the split-image screen 52 respectively, but not limited to this.

In fact, the light emitter 31 can also include an invisible light source, and the detection light adopts invisible light, and at least one light-emitting unit that can selectively control lighting or dimming is disposed at the staring point 53, such as a light-emitting diode, a lamp, light bulbs or optical fibers, but not limited to this.

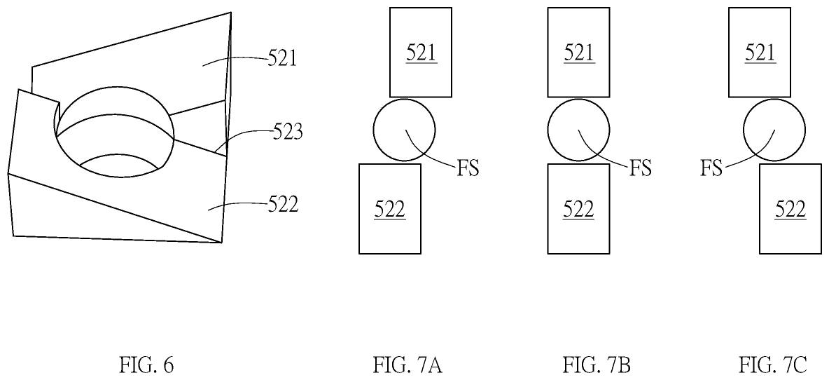

In this embodiment, as shown in FIG. 6, a light-transmitting space 523 is formed between the two prisms 521 and 522 disposed on the shield 520 of the split-image screen 52, and the light-transmitting space 523 is formed by separating the two prisms 521 and 522 from the middle of the two prisms 521 and 522, but not limited to this. As for FIG. 7A to FIG. 7C, FIG. 7A to FIG. 7C illustrate schematic diagrams showing that the two prisms 521 and 522 can have different relative positions respectively, wherein FS is the focus point.

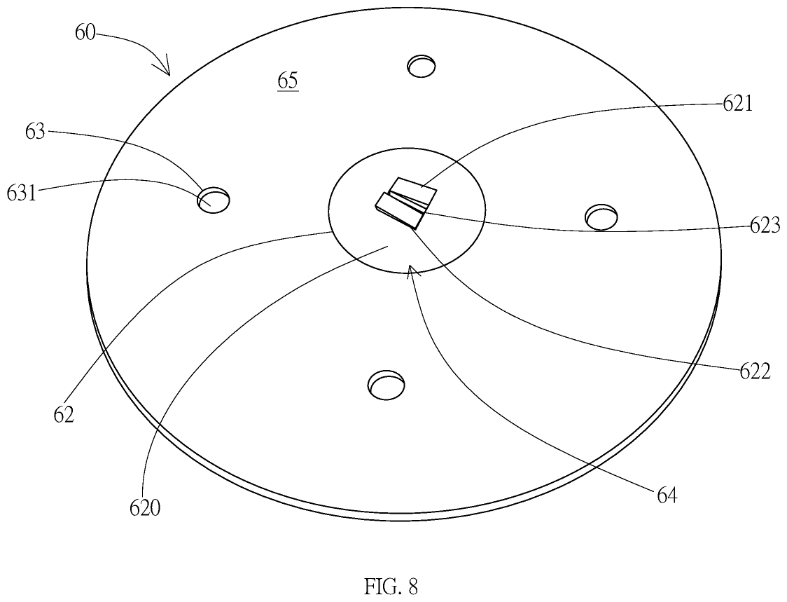

In the second preferred embodiment, as shown in FIG. 8, the auxiliary component 60 is provided with a split-image screen 62 and a plurality of staring points 63. The split-image screen 62 cooperates with the focusing lens 61 to form a focusing device generating split-image focusing, and the staring point 63 cooperates with the contrast of the detecting light to form a staring device to allow the subject to rotate his/her eyeball to a specific required angle in response to the displayed staring target.

The split-image screen 62 has a shutter 620 with a default size. Two prisms 621 and 622 opposite to each other are disposed on the shutter 620, and a light-transmitting space 623 for the central staring point to pass through is formed between the two prisms 621 and 622; and the focusing lens 51 is located between the relay lens 42 and the image display device 43 to form an adjustment means that can change the focal length position. Thus, the detection light can pass through the light-transmitting space 623, and then change the direction of the detection light through the two prisms 621 and 622, and generate two rectangular images on the image display 43 to indicate whether the focus is completed, but not limited to this.

Furthermore, the split-image screen 62 and the staring point 63 are located on the same staring surface to form an opaque area 64 shielding the lighting system 30, and a light-transmitting area 65 is formed by the blocks outside the opaque area 64. The opaque area 64 is made of opaque material to avoid being penetrated by the detection light, while the light-transmitting area 65 is made of transparent material, so that the detection light can be projected into the subject's eyes.

In this embodiment, the light emitter 31 includes a visible light source, the detection light adopts visible light, and the light-transmitting area 65 is set as a transparent substrate, and an opaque split-image screen 62 is disposed in the center of the transparent substrate. Four light-shielding units 631 are disposed at default distances outside the split-image screen 62 to form opaque staring points 63, wherein the staring points 63 are located at the upper, lower, left, and right directions outside the split-image screen 62 respectively, but not limited to this.

In fact, the light emitter 31 can also include an invisible light source, and the detection light adopts invisible light, and at least one light-emitting unit that can selectively control lighting or dimming is disposed at the staring point 63, such as a light-emitting diode, a lamp, light bulbs or optical fibers, but not limited to this.

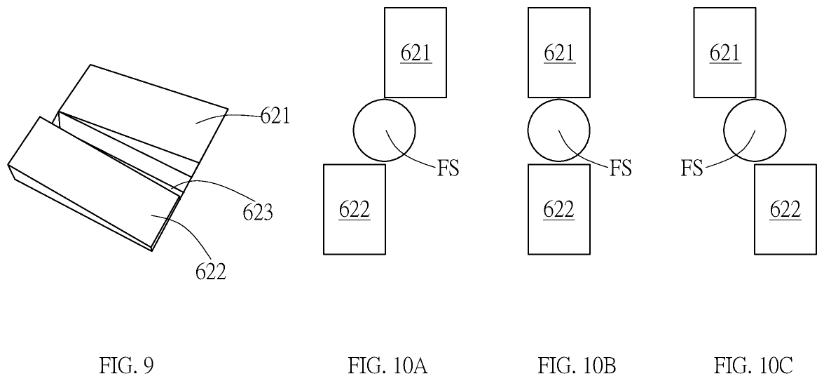

In this embodiment, as shown in FIG. 9, a light-transmitting space 623 for the central staring point to pass through is formed between the two prisms 621 and 622 disposed on the shutter 620 of the split-image screen 62, and the light-transmitting space 623 is formed by replacing the middle of the two prisms 621 and 622 with a plane, but not limited to this. As for FIG. 10A to FIG. 10C, FIG. 10A to FIG. 10C illustrate schematic diagrams showing the two prisms 621 and 622 with different relative positions respectively, wherein FS is the focus.

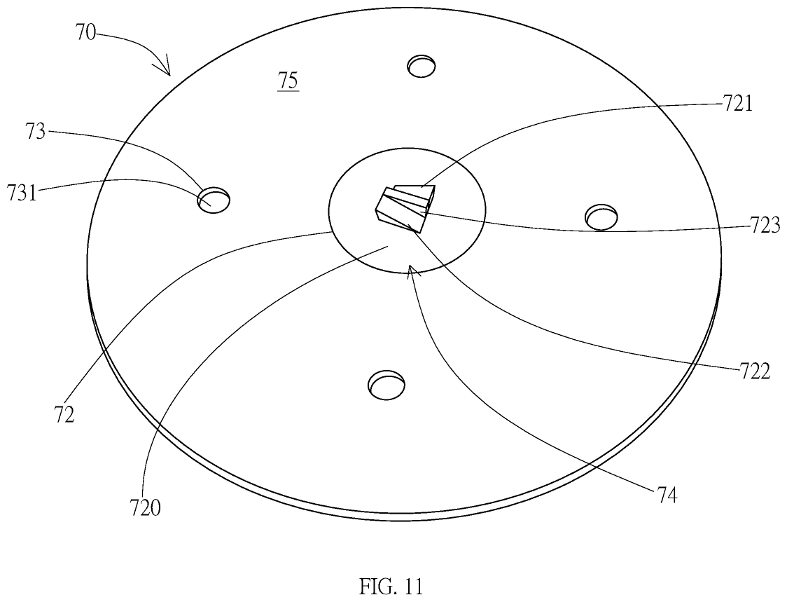

In the third preferred embodiment, as shown in FIG. 11, the auxiliary component 70 is provided with a split-image screen 72 and a plurality of staring points 73. The split-image screen 72 cooperates with the focusing lens 71 to form a focusing device generating split-image focusing, and the staring point 73 cooperates with the contrast of the detecting light to form a staring device to allow the subject to rotate his/her eyeball to a specific required angle in response to the displayed staring target.

The split-image screen 72 has a shutter 720 with a default size. Two prisms 721 and 722 opposite to each other are disposed on the shutter 720, and a light-transmitting space 723 for the central staring point to pass through is formed between the two prisms 721 and 722; and the focusing lens 51 is located between the relay lens 42 and the image display device 43 to form an adjustment means that can change the focal length position. Thus, the detection light can pass through the light-transmitting space 723, and then change the direction of the detection light through the two prisms 721 and 722, and generate two rectangular images on the image display 43 to indicate whether the focus is completed, but not limited to this.

Furthermore, the split-image screen 72 and the staring point 73 are located on the same staring surface to form an opaque area 74 shielding the lighting system 30, and a light-transmitting area 75 is formed by the blocks outside the opaque area 74. The opaque area 74 is made of opaque material to avoid being penetrated by the detection light, while the light-transmitting area 75 is made of transparent material, so that the detection light can be projected into the subject's eyes.

In this embodiment, the light emitter 31 includes a visible light source, the detection light adopts visible light, and the light-transmitting area 75 is set as a transparent substrate, and the opaque split-image screen 72 is disposed in the center of the transparent substrate. Four light-shielding units 731 are disposed at default distances outside the split-image screen 72 to form opaque staring points 73, wherein the staring points 73 are located at the upper, lower, left, and right directions outside the split-image screen 72 respectively, but not limited to this.

In fact, the light emitter 31 can also include an invisible light source, and the detection light adopts invisible light, and at least one light-emitting unit that can selectively control lighting or dimming is disposed at the staring point 73, such as a light-emitting diode, a lamp, light bulbs or optical fibers, but not limited to this.

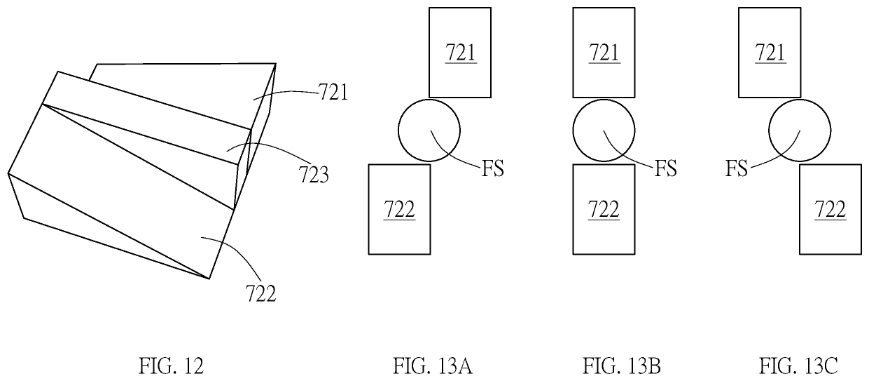

In this embodiment, as shown in FIG. 12, a light-transmitting space 723 for the central staring point to pass through is formed between the two prisms 721 and 722 disposed on the shutter 720 of the split-image screen 72, and the light-transmitting space 723 is formed by replacing the middle of the two prisms 721 and 722 with a plane, but not limited to this. As for FIG. 13A to FIG. 13C, FIG. 13A to FIG. 13C illustrate schematic diagrams showing the two prisms 721 and 722 with different relative positions respectively, wherein FS is the focus.

Compared to the prior art, the auxiliary staring and imaging focusing device proposed by the invention has the following advantages and effects:
(1) directly integrating the staring and focusing device into the lighting and imaging system without the need to extend other optical-path systems, so the optical-path structure design of the fundus camera can be greatly simplified;
(2) designing an independently controllable staring point as a staring target to allow the subject to focus more on staring in a specific direction; and
(3) the shutter of the split-image screen is provided with two prisms facing each other and a light-transmitting space is formed between the two prisms for a center staring point to pass through. Therefore, the shortcoming in the prior art that the central staring point cannot pass through the slit on the shutter of the split-image screen can be effectively improved.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. An auxiliary staring and imaging focusing device, comprising:
an illumination system having an illumination optical-path to project a detection light to illuminate a fundus of a subject's eye;
an imaging system having an imaging optical-path for receiving a reflected light and a fundus image of the subject and imaging the reflected light and the fundus image on an image display;
a staring device, located in the illumination optical-path, forming a staring surface provided with staring points contrast with a detection light for the subject to watch; and
a focusing device provided with a split-image screen in the illumination optical-path, the split-image screen having a shutter with a default size, and two facing prisms being disposed on the shutter and a light-transmitting space being formed between the two prisms for a center staring point to pass through.

2. The auxiliary staring and imaging focusing device of claim 1, wherein the light-transmitting space is formed by separating the two prisms from the middle of the two prisms.

3. The auxiliary staring and imaging focusing device of claim 1, wherein the light-transmitting space is formed by replacing the middle of the two prisms with a plane.

4. The auxiliary staring and imaging focusing device of claim 1, wherein the two prisms are made of plastic material, and the light-transmitting space is formed by digging a hole in the middle of the two prisms.

5. The auxiliary staring and imaging focusing device of claim 1, wherein the illumination system is provided with an additional light-emitting unit set as a light-emitting diode, a lamp, a light bulb or an optical fiber.

6. The auxiliary staring and imaging focusing device of claim 1, wherein the staring point and the split-image screen are located on the same staring surface, and the staring surface is set as a vertical plane, and the staring point is located at set positions in four directions of up, down, left and right outside the split-image screen.

7. The auxiliary staring and imaging focusing device of claim 1, wherein the staring point and the split-image screen are located on the same staring surface, and the staring surface is set as a concave surface corresponding to the curvature of the human eye, and the staring point is located on the concave surface in four directions of up, down, left and right in front of the split-image screen.

8. The auxiliary staring and imaging focusing device of claim 1, wherein the detection light is a visible light, and the staring point is provided with at least one opaque-type light-shielding unit.

9. The auxiliary staring and imaging focusing device of claim 8, wherein the split-image screen and the staring point jointly form an opaque area blocking the illumination optical-path, and a light-transmitting area is formed by blocks outside the opaque area.

10. The auxiliary staring and imaging focusing device of claim 9, wherein the light-transmitting area is provided with a transparent substrate, and the split-image screen is disposed in the center of the transparent substrate, and the light-shielding unit is disposed at a set position on the transparent substrate outside the split-image screen.

11. The auxiliary staring and imaging focusing device of claim 1, wherein the detection light is an invisible light, and the staring point is provided with at least one light-emitting unit that can selectively control lighting or dimming, the light-emitting unit is set as a light-emitting diode, a lamp, a light bulb or an optical fiber.
